(19) United States
(12) Patent Application Publication
(10) Pub. No.: US 2025/0179485 A1
(43) Pub. Date: Jun. 5, 2025

(54) ACTIVE TARGETING MICRORNA OLIGONUCLEOTIDE THERAPEUTICS FOR THE TREATMENT OF CARDIO-METABOLIC DISEASES

(71) Applicant: AptamiR Therapeutics, Inc., Naples, FL (US)

(72) Inventor: Marc THIBONNIER, Naples, FL (US)

(73) Assignee: AptamiR Therapeutics, Inc., Naples, FL (US)

(21) Appl. No.: 18/332,414

(22) Filed: Jun. 9, 2023

(51) Int. Cl. C12N 15/113

(52) U.S. Cl. CPC C12N 15/113; C12N 2310/113; C12N 2310/141; C12N 2310/3181; C12N 2310/3233; C12N 2310/3513; C12N 2310/3515

(57) ABSTRACT

Disclosed herein are novel methods and compositions for treatment of obesity and/or related cardio-metabolic disorders such as dyslipidemia, Type 2 diabetes mellitus, NASH, NAFLD, and/or MAFLD. In some embodiments, compositions may include a) a miRNA antagomir or agomir oligonucleotide therapeutic (ONT) agent capable of modulating lipid oxidation, mitochondrial activity, energy expenditure, fat accumulation, inflammation, and/or necrosis, and b) a targeting element facilitating cellular uptake and delivery of a miRNA ONT agent to targeted adipocytes and metabolic organs.

### SEQUENCE LISTING

The instant application contains a Sequence Listing which has been submitted in ST26 format and is hereby incorporated by reference in its entirety. Said ST26 copy, created on Nov. 8, 2023, is named APTA_P0011US_Sequence_Listing.xml and is 13,123 bytes in size.

### BACKGROUND

### I. Field

The immediate disclosure generally concerns at least compositions comprising oligonucleotide therapeutics (ONTs) such as microRNA agomirs and/or antagomirs, and methods for active targeted delivery of such therapeutic agents to adipocytes and metabolic organs. The provided compositions can be utilized in methods of treatment of human obesity and/or related cardiometabolic disorders, including but not limited to, insulin resistance, Type 2 diabetes mellitus, dyslipidemia, Non-Alcoholic Fatty Liver Disease (NAFLD), Non Alcoholic Steatohepatitis (NASH) and/or Metabolic Associated Fatty Liver Disease (MAFLD).

### II. Description of Related Art

Obesity and being overweight are growing global epidemics. Obesity affects one third of the world population, including millions of children. Obesity is the result of a chronic imbalance between energy intake and expenditure. The recent worsening of the obesity epidemic can be attributed to the combination of excessive consumption of energy-dense foods rich in saturated fats and sugars, and reduced physical activity. This combination leads to storage of excess energy in the form of triglycerides in adipocytes, which typically exhibit both hypertrophy (increase in cell size) and hyperplasia (increase in cell number or adipogenesis). These cellular changes result in fat accumulation, inflammation, and necrosis. Which in turn can lead to lipotoxicity, dyslipidemia, insulin resistance, diabetes, liver steatosis, inflammation, and/or fibrosis, as shown in FIG. 1, adapted from [1].

Current medical treatments of obesity fail to achieve their long-term therapeutic goals, mainly due to limited drug efficacy, patients' poor adherence with lifestyle changes and therapies, and/or poor insurance coverage. These drugs are characterized by a Mechanism of Action described as “one drug-one target”, or “one drug-two/three targets”. Several obesity drugs have been removed from the market for safety reasons. Presently, only restrictive and malabsorptive bariatric surgery can achieve significant long-term reduction of weight excess with some favorable cardiovascular benefits. However, bariatric surgery creates a state of chronic digestive malabsorption. Accordingly, there is a need in the art for novel treatments of obesity. Recent developments of Glucagon-like peptide-1 receptor agonists (GLP-1 RAs) suggest that a meaningful weight reduction can be achieved by a medical treatment [2-4].

NAFLD (Non Alcoholic Fatty Liver Disease) is a growing pandemic, affecting up to 31% of the adult human population worldwide, in relation with an increased prevalence of obesity and type 2 diabetes [5]. One hundred million Americans have a fatty liver, and most don't know it. Many individuals will develop NAFLD, NASH (Non Alcoholic SteatoHepatitis), liver fibrosis, possibly end stage liver failure, and/or cancer [6]. Without any currently approved therapy, there is a pressing need for safe, effective and convenient treatments for NAFLD/NASH/MAFLD. NAFLD/NASH/MAFLD are a major cause of chronic liver disease and are associated with substantial morbidity and mortality in developed countries [7]. NAFLD/NASH/MAFLD can be considered fairly complex diseases, and a “multiple-hit” hypothesis has been proposed to explain their development [8]: a) First hit: Lipid accumulation (steatosis); b) Second hit: inflammation, mitochondrial dysfunction and oxidative stress (steatohepatitis); and c) Third hit: defective hepatocyte regeneration (fibrosis).

MicroRNAs (miRNAs) are small non-coding RNAs (ncRNAs) that post-transcriptionally regulate genes. miRNA based post-transcriptional regulation can result in altered protein expression. miRNAs are attractive drug targets for regulating cell fate decisions and improving complex diseases, at least because the simultaneous modulation of many target genes by a single miRNA (e.g., a pleiotropic concept of “One Drug-Multiple Targets”) may provide effective therapies of multifactorial diseases like obesity, dyslipidemia, type 2 diabetes mellitus, and/or NAFLD/NASH/MAFLD. As the inventors previous work has shown, miRNAs are extensive regulators of adipocyte differentiation, development, and functions, and miRNAs are viable therapeutic agents/targets for combating obesity and/or disease states associated therewith (obesity-related diseases). Altered miRNAs expression has been reported in association with obesity, both in animal and human studies [9]. Dysregulation of miRNAs may alter the status and functions of various tissues and organs, including but not limited to, adipose tissues, the pancreas, the liver, and muscle tissues. Dysregulation of miRNAs in these tissues can lead to metabolic abnormalities associated with obesity and/or obesity-related diseases.

Several ONT miRNA agonists (agomirs) and antagonists (antagomirs) are currently in development to treat various human diseases [10, 11]. Examples of such agents are disclosed in U.S. Pat. No. 9,034,839 granted May 19, 2015 to Thibonnier, which is incorporated herein by reference in its entirety for the purposes described herein.

miRNA inhibitors (“antagomirs”) are single-stranded oligonucleotides that bind to complementary miRNAs through Watson-Crick base-pairing, blocking the interaction of miRNAs with target mRNAs. miRNA mimics (“agomirs”) are chemically modified single-stranded and double-stranded oligonucleotide versions of native miRNAs that can be loaded into the RNA-induced silencing complex (RISC) to bind and regulate target mRNAs via their “guide” strand while the complementary “passenger” strand is degraded. The mechanisms of action of miRNA antagomirs and agomirs are shown in FIG. 2, which was adapted from [12].

To improve the structure-activity relationship of miRNA ONTs, various chemical modifications can be introduced into their structure as shown in FIG. 3, which was adapted from [11].

The technology platform to transform oligonucleotides into drugs has recently matured and several drugs are now in clinical development [10, 12, 13]. However, targeted delivery of ONTs to tissues and organs besides the liver has been seldom tested, and the field could benefit from new and innovative approaches [14-20].

There exists a need to achieve an active targeted delivery of microRNA agomirs and antagomirs to adipocytes and/or metabolic organs in order to optimize their long-term efficacy/safety profile, improve their PK/PD profile with an extended mean residence time (MRT) inside the targeted tissues, minimize off-target effects, and/or reduce the cost of goods/therapeutic approaches [21].

### SUMMARY OF THE DISCLOSURE

In some embodiments, provided herein are compositions and methods for treating human obesity and/or related cardiometabolic disorders, including but not limited to, type 2 diabetes mellitus, dyslipidemia, Non-Alcoholic Fatty Liver Disease (NAFLD), Non Alcoholic Steatohepatitis (NASH), and/or Metabolic Associated Fatty Liver Disease (MAFLD). In some embodiments, provided herein are new and innovative combinations of: (a) Novel miRNA agomirs and/or antagomirs ONTs targeting specific miRNAs modulating mRNAs and proteins involved in lipid oxidation, mitochondrial activity, energy expenditure, fat accumulation, inflammation, and/or necrosis; and/or (b) targeting elements (e.g., molecules transported by the cellular membrane transporter Fatty Acid Translocase (FAT)) that facilitate cellular uptake and delivery of the miRNA agomirs and/or antagomirs inside the targeted adipocytes and/or metabolic organs.

In some embodiments, compositions that employ such ONT therapeutic agents and/or targeting elements can be used in methods employing local subcutaneous administration (e.g., injection, patch, or microneedles, etc.) of the therapeutic agents to the human adipose tissue and/or metabolic organs. In some embodiments, a targeted supply of ONT therapeutic agents results in minimization of systemic exposure and “off target effects,” further improving therapeutic index, reducing cost of goods, improving patients' convenience, and/or improving patients' adherence to treatment regimens.

In some embodiments, a therapeutic agent comprises a miRNA agomir and/or antagomir from 14 to 25 nucleotides in length.

In some embodiments, a therapeutic agent is targeting one or more “Metabolic” miRNAs as listed in Table 1.

TABLE 1
187 metabolic miRNAs (listed in alphabetic/ascending order)

| | | | | |
|---|---|---|---|---|
| let-7 | miR-152 | miR-216a | miR-33a | miR-512-2 |
| let-7c-5p | miR-155 | miR-217 | miR-33b | miR-515 |
| miR-1 | miR-1587 | miR-218 | miR-342 | miR-515-3p |
| miR-1-1 | miR-15a | miR-218.1 | miR-344 | miR-519c-3p |
| miR-1-2 | miR-15b | miR-218.2 | miR-34a | miR-519e |
| miR-103 | miR-16 | miR-219.2 | miR-3613-3p | miR-520a |
| miR-104 | miR-17/92 | miR-219.2-3p | miR-362 | miR-520a-3p |
| miR-105 | miR-181a | miR-22 | miR-362-5p | miR-520b |
| miR-106a-5p | miR-184 | miR-22-3p | miR-363 | miR-543 |
| miR-107 | miR-186 | miR-22-5p | miR-365 | miR-545 |
| miR-1207-5p | miR-187 | miR-221 | miR-3658 | miR-549 |
| miR-122 | miR-19 | miR-222 | miR-367 | miR-556 |
| miR-124a | miR-192 | miR-223 | miR-369 | miR-568 |
| miR-125a | miR-193a | miR-224 | miR-370 | miR-620 |
| miR-126 | miR-193b | miR-23a | miR-371 | miR-636 |
| miR-128-1 | miR-194 | miR-24 | miR-371-5p | miR-637 |
| miR-1283 | miR-195 | miR-24-2 | miR-375 | miR-643 |
| miR-129 | miR-196a | miR-25 | miR-377 | miR-654-3p |
| miR-1307-3p | miR-199a | miR-26a | miR-378 | miR-690 |
| miR-130a | miR-199b | miR-26b | miR-378a-5p | miR-7 |
| miR-130b | miR-19a | miR-27a/b | miR-382 | miR-709 |
| miR-132 | miR-19a-b | miR-28 | miR-383 | miR-765 |
| miR-133 | miR-200 | miR-298 | miR-422a | miR-7a-g |
| miR-133a-1 | miR-200a | miR-29a | miR-424 | miR-802 |
| miR-133a-2 | miR-200b | miR-29a | miR-425 | miR-871 |
| miR-135 | miR-200c | miR-29b | miR-448 | miR-888 |
| miR-138 | miR-204 | miR-29c | miR-451-1 | miR-888-3p |
| miR-141 | miR-205 | miR-30a-5p | miR-455 | miR-92b |
| miR-143 | miR-206 | miR-30b | miR-455-3p | miR-92b-3p |
| miR-143-5p | miR-208 | miR-30c | miR-455-5p | miR-93 |
| miR-144 | miR-20a | miR-30d | miR-466b | miR-93-5p |
| miR-145 | miR-20b | miR-3177-5p | miR-491 | miR-96 |
| miR-146b | miR-21 | miR-31a | miR-491-5p | miR-96/182 |
| | | | | cluster |
| miR-147 | miR-21-5p | miR-320 | miR-494 | miR-99a |
| miR-1470 | miR-210 | miR-325 | miR-508 | miR-99b |
| miR-148 | miR-211 | miR-330a-e | miR-508-5p | |
| miR-149 | miR-214 | miR-331 | miR-5089-3p | |
| miR-150-5p | miR-215-5p | miR-331-5p | miR-512-1 | |

In some aspects, a composition comprises, consists essentially of, or consists of an agomir and/or antagomir targeting a sequence of at least, at most, or exactly 14, 15, 16, 17, 18, 19, 20, 21, 22, 23, 24, or 25 nucleotides of one or more miRNAs of interest, including any combination thereof.

In some embodiments, a therapeutic agent comprises an oligonucleotide therapeutic (ONT) agent. In some embodiments, an oligonucleotide therapeutic comprises, consists essentially of, or consists of a single-stranded oligonucleotide miRNA antagomir and/or agomir, and/or a double-stranded oligonucleotide miRNA agomir.

In some embodiments, an oligonucleotide therapeutic comprises, consists essentially of, or consists of a miR-22 antagomir and/or a miR-515 agomir.

In some embodiments, a targeting element comprises, consists essentially of, or consists of a lipid specifically transported by the cellular membrane transporter Fatty Acid Translocase (FAT/CD36/SCARB3) [22].

In some embodiments, a targeting element (e.g., one or more lipids) is linked to the therapeutic agent. In some embodiments, a lipid comprises, consists essentially of, or consists of cholesterol, decanoic acid, dodecanoic acid, palmitic acid/hexadecanoic acid, stearic acid, oleic acid, docosanoic acid, dotriacontahexaenoic acid and/or docosahexaenoic acid (see e.g., Table 2). In some embodiments, a lipid comprises, consists essentially of, or consists of one or more medium chain fatty acids. In some embodiments, a lipid comprises, consists essentially of, or consists of one or more long chain fatty acids. In some embodiments, a lipid comprises, consists essentially of, or consists of one or more very long chain fatty acids. In some embodiments, a lipid comprises, consists essentially of, or consists of one or more omega-3 fatty acids.

TABLE 2
Exemplary Lipids

| | |
|---|---|
| Medium Chain Fatty Acids | C10:0 Decanoic Acid |
| | C12:0 Dodecanoic Acid |
| Long Chain Fatty Acids | C16:0 Palmitic Acid |
| | C18:0 Stearic Acid |
| | C18:1 Oleic Acid |
| Very Long Chain Fatty Acids | C22:0 Docosanoic acid |
| | C32:6 Dotriacontahexaenoic Acid |
| Omega-3 Fatty Acids | C22:6 Docosahexaenoic acid |

In some embodiments, a therapeutic agent is linked to the lipid by one or more linkers selected from the group consisting of a covalent bond, a disulfide bond, a diester bond, a peptide bond, an ionic bond, and a biotin-streptavidin bond.

In some embodiments, a targeting element is a peptide specifically transported by the cellular membrane transporter FAT.

In some embodiments, a peptide is linked to the therapeutic agent. In some embodiments, the peptide comprises, consists essentially of, or consists of: Hexarelin (His-D-2-Me-Trp-Ala-Trp-D-Phe-Lys-NH2) (SEQ ID NO: 4), thrombospondin 1 (TSP-1), a TSP-1 peptide having the amino acid sequence GVITRIR (SEQ ID NO: 1) and/or VTCGVITRIR (SEQ ID NO: 2), and/or a Prohibitin (PHB) peptide having the amino acid sequence CKGGRAKDC (SEQ ID NO: 3), all of which can also be transported by the cellular membrane transporter FAT [23, 24].

In some embodiments, a therapeutic agent is linked to the targeting element by a linker selected from the group consisting of a covalent bond, a disulfide bond, a diester bond, a peptide bond, an ionic bond, and a biotin-streptavidin bond.

In some embodiments, a therapeutic agent modulates mRNAs and/or proteins involved in lipid oxidation, mitochondrial activity, energy expenditure, fat accumulation, inflammation, and/or necrosis.

In some embodiments, a disease and/or condition is obesity, and/or a cardiometabolic disorder such as but not limited to type 2 diabetes mellitus, dyslipidemia, and/or NAFLD/NASH/MAFLD.

In some embodiments, a patient receiving a composition described herein has, and/or has been diagnosed with, obesity, and/or a related cardiometabolic disorder such as type 2 diabetes mellitus, dyslipidemia, and/or NAFLD/NASH/MAFLD.

Certain embodiments of the present invention(s) are characterized through the following aspects.

Aspect 1 is a composition comprising, a) one or more miRNA oligonucleotide therapeutic (ONT) agent(s); and b) one or more targeting element(s) linked to the ONT by a linker, wherein the one or more targeting element(s) facilitate active targeted cellular uptake and/or delivery of the ONT agent to adipocytes and/or metabolic organs.

Aspect 2 is the composition of aspect 1, wherein the one or more ONT agent(s) comprise or consist essentially of miRNA agomirs, and/or antagomirs targeting specific miRNAs.

Aspect 3 is the composition of aspect 1 or 2, wherein the one or more ONT agent(s) are 14 to 25 nucleotides in length.

Aspect 4 is the composition of any one of aspects 1 to 3, wherein one or more of the ONT agent(s) comprise a peptide nucleic acid (PNA) backbone, optionally wherein the PNA is a gamma PNA.

Aspect 5 is the composition of any one of aspects 1 to 4, wherein the ONT agent comprises a miR-22 antagomir and/or a miR-515 agomir.

Aspect 6 is the composition of any one of aspects 1-5, wherein the ONT agent comprises a miR-22 antagomir according to SEQ ID NO: 12 (5′-CTTCTTCAACTGGCAGCT-3′), and/or a miR-515 agomir according to SEQ ID NO: 13 (5′-GAGUGCCUUCUUUUGGAGCGUU-3′).

Aspect 7 is the composition of any one of aspects 1 to 6, wherein the one or more targeting element(s) comprise a lipid.

Aspect 8 is the composition of aspect 7, wherein the lipid comprises or consists essentially of, cholesterol, decanoic acid, dodecanoic acid, palmitic acid, stearic acid, oleic acid/hexadecanoic acid, oleoyl glycine, docosanoic acid, dotriacontahexaenoic acid, and/or docosahexaenoic acid.

Aspect 9 is the composition of aspect 7 or 8, wherein the lipid comprises a fatty acid that is transported by the membrane transporter Fatty Acid Translocase (FAT)).

Aspect 10 is the composition of any one of aspects 1 to 9, wherein the ONT agent is linked to one or more targeting elements by a linker selected from the group consisting of a covalent bond, a disulfide bond, a diester bond, a peptide bond, an ionic bond, and a biotin-streptavidin bond.

Aspect 11 is the composition of any one of aspects 1 to 10, wherein a linker is a cleavable linker.

Aspect 12 is the composition of any one of aspects 1 to 11, wherein one or more targeting element(s) specifically bind to the membrane transporter FAT.

Aspect 13 is the composition of any one of aspects 1 to 12, wherein the one or more targeting element(s) comprises a peptide.

Aspect 14 is the composition of aspect 13, wherein the peptide comprises hexarelin (SEQ ID NO: 4), Thrombospondin-1 (TSP-1), a TSP-1 peptide having the amino acid sequence GVITRIR (SEQ ID NO: 1) and/or VTCGVITRIR (SEQ ID NO: 2), and/or a Prohibitin (PHB) peptide having the amino acid sequence CKGGRAKDC (SEQ ID NO: 3).

Aspect 15 is the composition of any one of aspects 12 to 14, wherein the one or more ONT agent(s) is linked to a peptide targeting element by a linker selected from the group consisting of a covalent bond, a disulfide bond, a diester bond, a peptide bond, an ionic bond, and a biotin-streptavidin bond.

Aspect 16 is the composition of any one of aspects 12 to 15, wherein a linker is a cleavable linker.

Aspect 17 is the composition of any one of aspects 1 to 16, wherein the one or more ONT agent(s) modulate lipid oxidation, mitochondrial activity, energy expenditure, fat accumulation, inflammation, and/or necrosis.

Aspect 18 is a method of treating a subject comprising administration of the composition according to any one of aspects 1 to 17.

Aspect 19 is the method of aspect 18, wherein administering the composition comprises subcutaneous, transcutaneous, and/or intravenous administration.

Aspect 20 is the method of aspect 18 or 19, wherein the subject has or is at risk of developing obesity and/or a cardio-metabolic disorder.

Aspect 21 is the method of aspect 20, wherein the subject has or is at risk of developing dyslipidemia, Type 2 diabetes mellitus, Non-Alcoholic Fatty Liver Disease (NAFLD), Non Alcoholic Steatohepatitis (NASH), and/or Metabolic Associated Fatty Liver Disease (MAFLD).

### BRIEF DESCRIPTION OF THE DRAWINGS

FIG. 1 depicts the relationships between fat accumulation, inflammation and necrosis resulting in lipotoxicity, dyslipidemia, insulin resistance, Type 2 diabetes mellitus, liver steatosis, inflammation and fibrosis (adapted from [25]).

FIG. 2 depicts the mechanisms of action of miRNA antagomirs and agomirs (adapted from [12]).

FIG. 3 illustrates the various chemical modifications introduced to improve the PK/PD profile of ONTs (adapted from [11]).

FIG. 4 shows the prediction of protein-protein interaction networks related to miR-22 using the protein-protein interaction-networks functional enrichment analysis tool String (https://string-db.org) (adapted from [25]).

FIG. 5 shows the histologic appearance of livers (H&E staining) at the end of 12 weeks of treatment in mice receiving SC injections of saline or the APT-110 miR-22-3p antagomir (two samples from each group were shown; adapted from [26]).

FIG. 6 shows the 2-D structure of the membrane transporter Fatty Acid Translocase (FAT) (adapted from [27]).

FIG. 7 shows the chemical structure of a Gamma PNA backbone.

FIGS. 8A-8C show the graphic representation of an 18-mer miR-22-3p antagomir (5′-CTTCTTCAACTGGCAGCT-3′) (SEQ ID NO: 12) with a PNA backbone coupled with (8A) C16 Palmitic Acid, (8B) C22 Docosanoic Acid, and (8C) C32:6 Dotriacontahexaenoic Acid.

FIG. 9 shows the graphic representation of an 18 mer miR-22-3p antagomir with a PNA backbone coupled with the hexapeptide Hexarelin.

FIG. 10 shows conservation of the human KDM3A 3′ UTR 29-35 region (SEQ ID NO: 5), KDM6B 3′ UTR 127-133 region (SEQ ID NO: 6), PPARA 3′ UTR 573-579 region (SEQ ID NO: 7), PPARGC1B 3′ UTR region 3393-3399 (SEQ ID NO: 8), PPARGC1B 3′ UTR region 5265-5271 (SEQ ID NO: 9), and SIRT1 3′ UTR region 530-537 (SEQ ID NO: 10), as conserved targets for hsa-miR-22-3p (SEQ ID NO: 11), produced using TargetScanHuman 8.0, www.targetscan.org/vert_80 (adapted from reference 31).

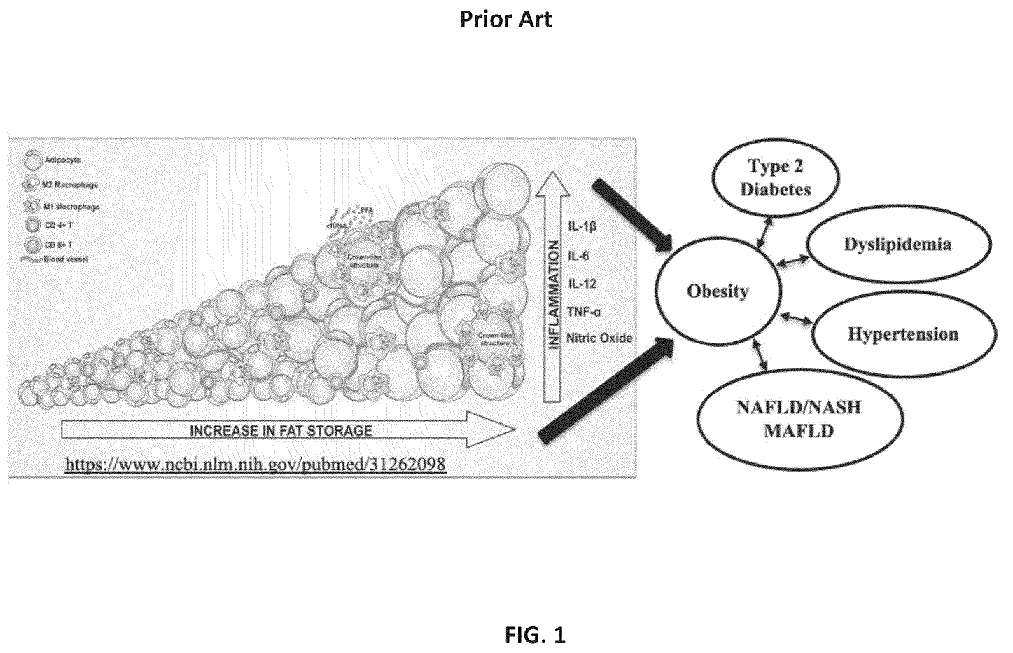

### DETAILED DESCRIPTION OF THE INVENTION

### I. DEFINITIONS

Unless otherwise defined, all technical and scientific terms used herein have the same meaning as commonly understood by one of ordinary skill in the art to which this invention belongs. In case of conflict, the present application, including definitions, will control.

In keeping with long-standing patent law convention, the words “a” and “an” when used in the present specification in concert with the word comprising, including the claims, denote “one or more.” Some embodiments of the disclosure may consist of, or consist essentially of, one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, the term “miRNA ONT” refers to an oligonucleotide antagomir or oligonucleotide agomir that directly or indirectly reprograms mesenchymal stem cells (ATMSCs) and/or white adipocytes (WAT) to become brown adipocytes (BAT), and/or modulates lipid oxidation, mitochondrial activity, energy expenditure, fat accumulation, inflammation and/or necrosis. miRNA ONTs can act on a target gene or an activator or repressor of a target gene, and/or on a target miRNA that directly or indirectly modulates the activity of a thermogenic regulator (e.g., a mitochondrial uncoupler or an activator or repressor thereof, etc.), and/or modifies lipid oxidation, mitochondrial activity, energy expenditure, fat accumulation, inflammation, and/or necrosis.

As used herein, the term “miRNA” refers to a single-stranded RNA molecule (or a synthetic derivative thereof), which is capable of binding to a target gene (e.g., a pre-mRNA, an mRNA, DNA, etc.) and regulating expression of that gene. In certain embodiments, an miRNA may be naturally expressed in an organism.

As used herein, the term “seed sequence” refers to a 6-8 nucleotide (nt) long substring within the first 8 nt at the 5′-end of the miRNA (i.e., seed sequence) that is an important determinant of target specificity.

As used herein, the term “agomir” refers to a synthetic oligonucleotide and/or oligonucleotide mimetic that functionally mimics a miRNA. An agomir can be an oligonucleotide with the same or similar nucleic acid sequence to a miRNA or a portion of a miRNA. In certain embodiments, an agomir can have 1, 2, 3, 4, 5, 6, 7, 8, 9 or 10 nucleotide differences from the miRNA that it mimics. Further, in some embodiments, agomirs can have the same length, a longer length, or a shorter length than the miRNA that they mimic. In certain embodiments, an agomir has the same 6-8 nucleotide sequence (e.g., seed sequence) at it's 5′ end as does of the miRNA it mimics. An agomir can be 14, 15, 16, 17, 18, 19, 20, 21, 22, 23, 24 or 25 nucleotides in length. In certain embodiments, agomirs can include any of the sequences shown in miRBase and/or other miRNA databases. In certain embodiments, chemically modified synthetic RNA duplexes include a guide strand that is identical or substantially identical to the miRNA of interest, e.g., to allow efficient loading into the RISC complex, whereas the passenger strand is chemically modified to prevent its loading to the Argonaute protein in the RISC complex (see e.g., Thorsen S B et al., Cancer J., 18(3):275-284 (2012); Broderick J A et al., Gene Ther., 18(12):1104-1110 (2011), each of which are incorporated herein in their entirety for the purposes described herein).

As used herein, the term “antagomir” refers to a synthetic oligonucleotide and/or oligonucleotide mimetic having complementarity to a specific miRNA, and which can inhibit the activity of that miRNA. The term “antimir” is synonymous with the term “antagomir”. In certain embodiments, an antagomir can have 1, 2, 3, 4, 5, 6, 7, 8, 9 or 10 nucleotide differences from the miRNA that it inhibits. Further, in some embodiments, antagomirs can have the same length, a longer length, or a shorter length than the miRNA that it inhibits. In certain embodiments, the antagomir hybridizes to 6-8 nucleotides at the 5′ end of the miRNA it inhibits (e.g., hybridizes to the seed sequence). An antagomir can be 14, 15, 16, 17, 18, 19, 20, 21, 22, 23, 24 or 25 nucleotides in length. In certain embodiments, antagomirs include oligonucleotides that are at least partially (e.g., greater than or equal to 20%, 25%, 30%, 35%, 40%, 45%, 50%, 55%, 60%, 65%, 70%, 75%, 80%, 85%, 90%, 95%, or 100%, or any range derivable therein) complementary to any one or more of the sequences shown in miRBase and/or other miRNA databases. In certain embodiments, antagomirs are synthetic reverse complements that tightly bind to and inactivate a specific miRNA. Various chemical modifications can be used to improve antagomir nuclease resistance and/or miRNA binding affinity. In some embodiments, a modification(s) to increase potency (e.g., improve nuclease resistance and/or miRNA binding affinity) can include, but are not limited to, various 2′-sugar modifications, such as 2′-O-Methyl (2′-O-Me), 2′-O-methoxyethyl (2′-MOE), and/or 2′-fluoro (2′-F). In certain embodiments, the nucleic acid structure of a miRNA antagomir and/or agomir can also be modified into a locked nucleic acid (LNA) with a methylene bridge between the 2′ oxygen and the 4′ carbon to lock the ribose in the 3′-endo (North) conformation in the A-type conformation of nucleic acids (see e.g., Lennox K A et al., Gene Ther. December 2011;18(12):1111-1120; Bader A G et al. Gene Ther. December 2011;18(12):1121-1126, which is incorporated herein in its entirety for the purposes described herein). In some embodiments, such a LNA modification can significantly increase target specificity and/or hybridization properties of the molecules. In some embodiments, additional and/or alternative modifications include but are not limited to, 5′-(E)-Vinylphosphonate protection (5′-VP), backbone modifications (e.g., phosphorothioate (PS)), Peptide Nucleic Acid (PNA), Phosphorodiamidate Morpholino Oligonucleotide (PMO), Ethylene-bridged Nucleic Acid (ENA), 5-Methylcytosine modification, introduction of a “pyrimidine cassette”, and/or introduction of a “DNA gap”.

As used herein, the term “interfering RNA” refers to any double stranded and/or single stranded RNA sequence capable of inhibiting and/or down regulating gene expression by mediating RNA interference. Interfering RNAs, include are not limited, to small interfering RNA (“siRNA”) and small hairpin RNA (“shRNA”). “RNA interference” refers to the selective degradation of a sequence-compatible messenger RNA transcript.

As used herein, the term “small interfering RNA” or “siRNA” refers to any small RNA molecule capable of inhibiting and/or down regulating gene expression by mediating RNA interference in a sequence specific manner. The small RNA can be, for example, about 14 to 25 nucleotides long.

As used herein, the term “shRNA” (small hairpin RNA) refers to an RNA molecule comprising an antisense region, a loop portion and a sense region, wherein the sense region has complementary nucleotides that base pair with the antisense region to form a duplex stem. Following post-transcriptional processing, the small hairpin RNA is converted into a small interfering RNA (siRNA) by a cleavage event mediated by the enzyme Dicer, which is a member of the RNase III family.

As used herein, the term “antisense oligonucleotide” (ASO) refers to a synthetic oligonucleotide and/or oligonucleotide mimetic that is complementary to specific RNA sequence.

As used herein, the term “miR-mask” refers to a single stranded antisense oligonucleotide that is complementary to a miRNA binding site in a target RNA (e.g., mRNA, etc.), and that serves to inhibit the binding of miRNA to the RNA binding site (see, e.g., Xiao, et al. “Novel approaches for gene-specific interference via manipulating actions of microRNAs: examination on the pacemaker channel genes HCN2 and HCN4,” Journal of Cellular Physiology, vol. 212, no. 2, pp. 285-292, 2007, which is incorporated herein in its entirety for the purposes described herein).

As used herein, the term “miRNA sponge” refers to a synthetic nucleic acid (e.g. a mRNA transcript) that contains multiple tandem-binding sites for a miRNA of interest, and that serves to titrate out an endogenous miRNA of interest, thus inhibiting the binding of the miRNA of interest to its endogenous targets (see, e.g., Ebert et al., “MicroRNA sponges: competitive inhibitors of small RNAs in mammalian cells,” Nature Methods, vol. 4, no. 9, pp. 721-726, 2007, which is incorporated herein in its entirety for the purposes described herein).

As used herein, the term “respiratory chain uncoupling” refers to the dissipation of the mitochondrial inner membrane proton gradient, thereby preventing the synthesis of ATP in the mitochondrion by oxidative phosphorylation.

As used herein, the term “mitochondrial uncoupler” refers to a protein (or the encoding nucleic acid) that can dissipate of the mitochondrial inner membrane proton gradient, thereby preventing the synthesis of ATP in the mitochondrion by oxidative phosphorylation. Exemplary mitochondrial uncouplers include without limitation, UCP1, UCP2, and UCP3.

As used herein, the terms “activator” or “repressor” of a mitochondrial uncoupler refers to a protein that serves to upregulate or downregulate, respectively, an activity of a mitochondrial uncoupler.

As used herein, the term “thermogenic regulator” refers to a therapeutic agent (e.g., oligonucleotide, small molecule, peptide, peptidomimetic, gene editing system, etc.) that regulates thermogenesis directly and/or indirectly. The term encompasses mitochondrial uncouplers and also activators and repressors of mitochondrial uncouplers.

As used herein, the term “modulate” refers to increasing or decreasing a parameter. For example, to modulate the activity of a protein, that protein's activity could be increased or decreased.

As used herein, the term “activity” of mitochondrial uncoupler or thermogenic regulator refers to any measurable biological activity including, without limitation, mRNA expression, protein expression, or respiratory chain uncoupling.

The “effective amount” of a composition or therapeutic agent is an amount sufficient to be effective in treating or preventing a disorder or to regulate a physiological condition in humans. In some embodiments, the disorder is obesity, dyslipidemia, Type 2 Diabetes mellitus, Non-Alcoholic Fatty Liver Disease (NAFLD), Non Alcoholic Steatohepatitis (NASH), and/or Metabolic Associated Fatty Liver Disease (MAFLD).

As used herein, “cardio-metabolic disorders” include at least cardiovascular diseases, stroke, hypertension, Type 2 diabetes mellitus, dyslipidemia, metabolic syndrome, Non-Alcoholic Fatty Liver Disease (NAFLD), Non Alcoholic Steatohepatitis (NASH), and Metabolic Associated Fatty Liver Disease (MAFLD).

A “subject” is a vertebrate, including any member of the class Mammalia, including humans, domestic and farm animals, and zoo, sports or pet animals, such as mouse, rabbit, pig, sheep, goat, cattle and higher primates.

The term “mammal” refers to any species that is a member of the class Mammalia, including rodents, primates, dogs, cats, camelids and ungulates. The term “rodent” refers to any species that is a member of the order rodentia including mice, rats, hamsters, gerbils and rabbits. The term “primate” refers to any species that is a member of the order primates, including monkeys, apes and humans. The term “camelids” refers to any species that is a member of the family camelidae including camels and llamas. The term “ungulates” refers to any species that is a member of the superorder Ungulata including cattle, horses and camelids. According to some embodiments, the mammal is a human.

“Treatment”, or “treating” as used herein, is defined as the application or administration of a therapeutic agent (e.g., oligonucleotide therapeutic, etc.) to a patient/subject, and/or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, and/or to improve or affect the disease or disorder, symptoms associated with the disease or disorder, and/or predisposition towards the disease or disorder.

“Pharmacogenomics”, as used herein, refers to the application of genomics technologies such as gene sequencing, statistical genetics, and/or gene expression analysis to drugs in clinical development and/or on the market. More specifically, the term refers to the study of how a patient's genes determine their response to a drug (e.g., a patient's “drug response phenotype”, or “drug response genotype”).

### II. REGULATION OF GENE EXPRESSION BY MICRORNAS

MicroRNAs (miRNAs) are small non-coding RNAs that bind to complementary RNAs, such as messenger RNAs (mRNAs) and subsequently regulate the RNA (e.g., mRNAs) and encoded proteins expression [28]. Natural miRNAs were evolutionarily selected to modulate the expression of select gene pathways. Natural miRNAs are synthesized as long single-stranded RNAs (pri-miRNA) that fold into hairpin loop structures (pre-miRNA). These hairpins are processed by the enzymes drosha and dicer into double-stranded mature miRNAs. The guide strand complementary to target mRNA transcripts is loaded into Argonaute (AGO) proteins while the passenger strand is removed [29]. The guide strand/AGO complex then binds by sequence complementarity to targets, these sequences are typically located within 3′-untranslated regions (3′-UTR) of mRNAs, but are not limited thereto.

In some embodiments, miRNA inhibitors (antagomirs) are engineered single-stranded oligonucleotides that bind to complementary miRNAs through Watson-Crick base-pairing, blocking their interaction with target mRNAs. In certain embodiments, to improve the structure-activity relationship of miRNA inhibitors, chemical modifications may be implemented. In some embodiments, chemical modifications may include any one or more modifications described herein. In some embodiments, chemical modifications include replacing: the phosphates in the oligonucleotide backbone with phosphorothioates (e.g., to inhibit nuclease degradation and/or promote plasma protein binding), such a modification can extend circulation time and/or tissue distribution. In some embodiments, chemical modifications include modifications to the 2′ carbon of the sugar group (2′-Fluor, 2′-O-methyl, 2′-methoxyethyl), and/or Locked Nucleic Acid (LNA) conformations. In some embodiments, 2′ carbon modifications and/or LNA conformations can inhibit nuclease degradation, increase affinity to target RNAs, and/or blunt the immune response to foreign nucleotides (e.g., DNA and/or RNA) [13]. In some embodiments, phosphorothioate backbone modified oligonucleotides can be administered subcutaneously in saline without additional formulation (e.g., the so called “naked miRNA inhibitors”), and generally have similar and predictable pharmacokinetics. In some embodiments, the initial distribution of phosphorothioate backbone modified oligonucleotides can be rapid, with a circulating t_{1/2} of a few hours, and a bioavailability over 90%. In some embodiments, phosphorothioate backbone modified oligonucleotides delivery to the liver, kidney, adipose tissue, spleen, and bone marrow is robust, with much lower amounts found in heart and muscle. In some embodiments, phosphorothioate backbone modified oligonucleotides compounds do not cross the blood-brain barrier.

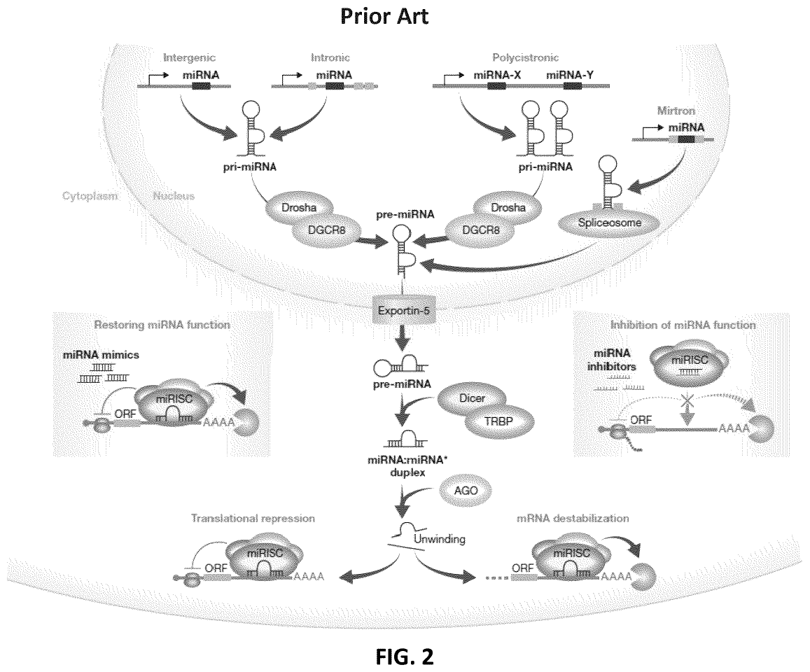

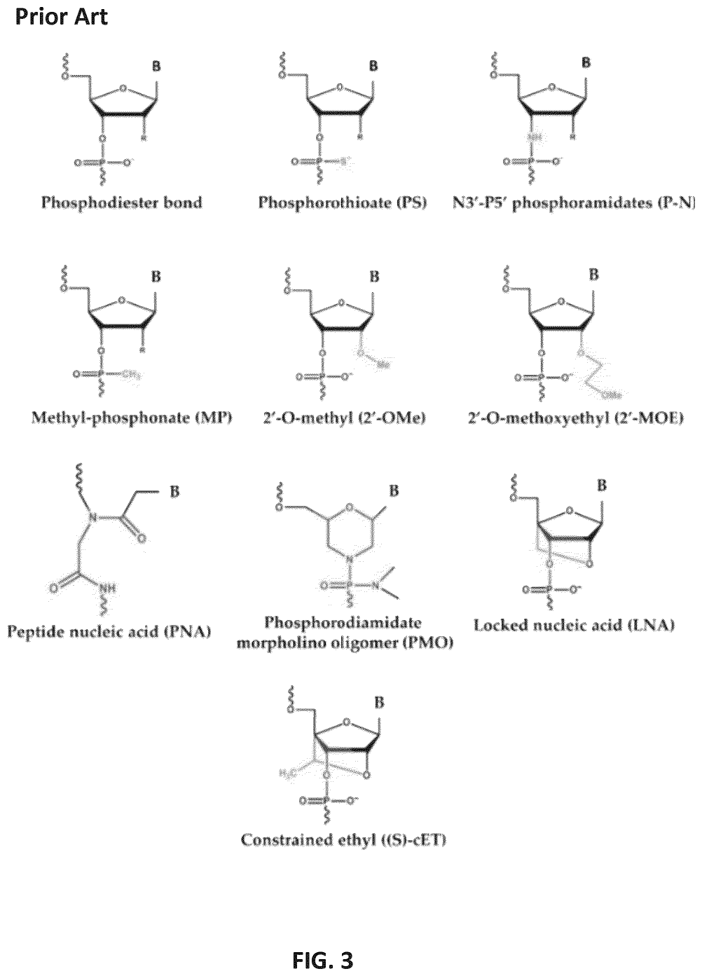

In some embodiments, miRNA mimics (agomirs) are chemically modified versions of native miRNAs that can be loaded into the RISC complex to bind and regulate target mRNAs via their “guide” strand while the complementary “passenger” strand can be degraded. In some embodiments, chemical modifications are used to protect the miRNA mimic from nuclease degradation and/or to improve potency. In some embodiments, the patterns of optimal chemical modification to an agomir can be different from siRNA and/or from single-stranded miRNA inhibitors. In some embodiments, synthetic chemically modified single-stranded miRNAs (ss-miRNAs) can mimic the functions of double-stranded miRNAs to silence the expression of target genes, as illustrated in FIG. 2 [30, 31]. In some embodiments, such action requires the recruitment of the Argonaute 2 (AGO2) protein to the target transcripts. In some embodiments, modified ss-miRNA mimics can combine the power of function through the RNAi pathway with the more favorable pharmacological properties of single stranded oligonucleotides. In some embodiments, in vivo effects of ss-miRNAs in animals can be achieved after systemic and/or local administration [30, 32, 33]. In some embodiments, as described herein, the inventors have developed targeting strategies that effectively deliver single-and/or double-stranded RNAs (e.g., agomirs and/or antagomirs) to adipocytes and/or metabolic organs.

### III. SELECTION OF MIR-22-3P AS A METABOLIC MIRNA

The inventors conducted an initial in silico analysis, which identified putative metabolic miRNAs from a known pool of about 2,000 miRNAs and a digitally curated list of 721 genes involved in lipid metabolism, oxidative phosphorylation, mitochondrial functions, respiratory cycle, browning of adipocytes, and energy expenditure. AptamiR 721 genes were selected using eight publicly available in silico tools, namely BioCarta; Database for Annotation, Visualization and Integrated Discovery (DAVID); GeneOntology; Gene Set Enrichment Analysis (GSEA); Kyoto Encyclopedia of Genes and Genomes (KEGG); PubGene; Reactome; and STRING.

The inventors then utilized 34 in silico miRNA target prediction tools and the proprietary in silico meta-tool (R-AptamiR) to identify 200 miRNAs that potentially bind to these target metabolic genes. In some embodiments, a metabolic target of particular interest is the mitochondrial uncoupling protein UCP1, which increases thermogenesis in adipose tissues.

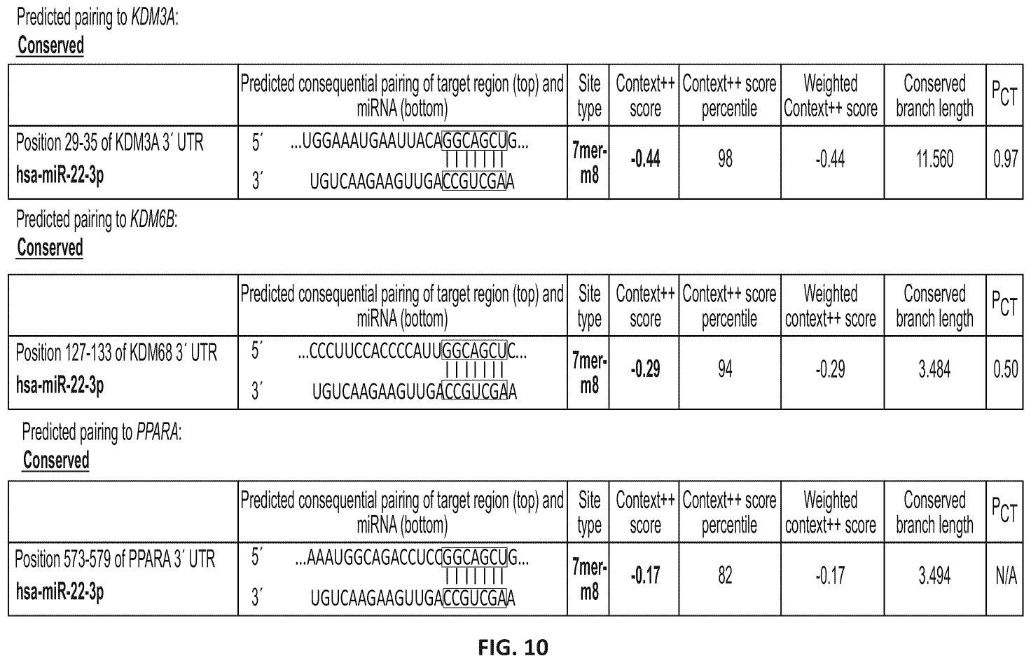

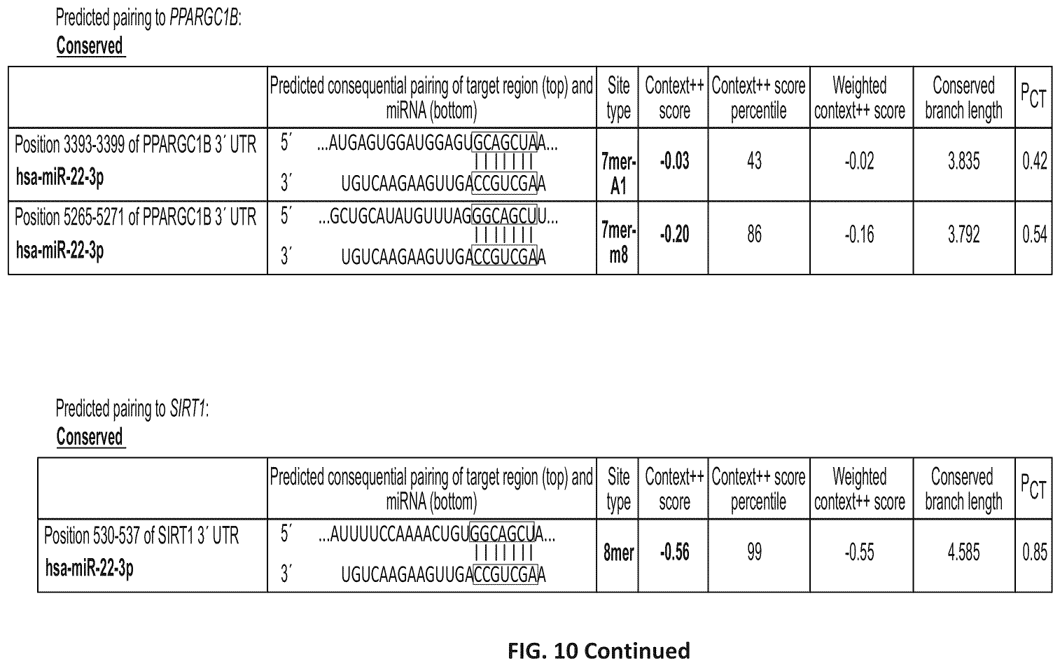

The human UCP1 gene structure is notable for a high degree of methylation (“CG islands”) in its promoter region. Methylation of CG islands within gene promoters can lead to their silencing [34]. The human lysine (K)-specific demethylase 3A (KDM3A) has been shown to be critically important in regulating the expression of metabolic genes and obesity resistance [35]. Using microarray technology, Zhang et al., demonstrated that a significant number of the genes involved in PPAR signaling and fatty acid oxidation (e.g., PPARA, ACADM, ACADL, ACADVL, AQP7) were down-regulated in response to KDM3A knockout [36]. KDM3A has been shown to directly regulate peroxisome proliferator-activated receptor alpha (PPARA) and UCP1 expression [37]. After a number of cycles of in silico, in vitro, and then in vivo experiments, the inventors identified a subset of miRNA targets from the original 200 that could function as metabolic regulators (e.g., Metabolic miRNA). The inventors found that the human KDM3A 3′ UTR 29-35 region (SEQ ID NO: 5) was a conserved target for hsa-miR-22-3p (SEQ ID NO: 11), as shown in FIG. 10, produced using TargetScanHuman 8.0, www.targetscan.org/vert_80. The inventors also found that the human KDM6B 3′ UTR 127-133 region (SEQ ID NO: 6), PPARA 3′ UTR 573-579 region (SEQ ID NO: 7), PPARGC1B 3′ UTR region 3393-3399 (SEQ ID NO: 8), PPARGC1B 3′ UTR region 5265-5271 (SEQ ID NO: 9), and SIRT1 3′ UTR region 530-537 (SEQ ID NO: 10) were also conserved targets for hsa-miR-22-3p (SEQ ID NO: 11), as shown in FIG. 10.

Using various in silico, in vitro and in vivo tools, the inventors demonstrated that miR-22-3p functioned as an excellent metabolic target that modulated several genes involved in lipid oxidation, mitochondrial activity, energy expenditure, fat accumulation, inflammation, and/or necrosis. Furthermore, the inventors found that miR-22-3p functioned as a unique, conserved, and universal miRNA that was highly expressed in adipose tissues and metabolic organs.

Using the in silico metaMIR tool (http://rna.informatik.uni-freiburg.de) which ranked miRNAs in relation to gene networks, the inventors found that hsa-miR-22-3p scored the highest in terms of interactions with clusters of metabolic genes of interest, as shown below in Table 3.

TABLE 3
in silico miRNA gene network regulation

| miRNA | Final Score | Positive Combo |
|---|---|---|
| hsa-miR-22-3p | 12.11 | AKT1, BDNF, CDKN1A, CREB1, ESR1, HDAC4, |
| | | HDAC6, KDM3A, KDM6B, KLF6, MAPK14, MECP2, |
| | | PPARA, PPARGC1B, PRDM16, PTEN, RUNX2, SIRT1, |
| | | SOD2, SP1, STAT3 |
| hsa-miR-1587 | 3.42 | AKT1, BDNF, CREB1, ESR1, HDAC6, KDM6B, |
| | | MAPK14, PPARGC1B, PRDM16, RUNX2, SOD2, SP1, |
| | | STAT3 |
| hsa-miR-30a-5p | 4.1 | AKT1, BDNF, CREB1, KDM3A, KDM6B KLF6, |
| | | PPARGC1B, PRDM16, PTEN, RUNX2, SIRT1, SOD2 |
| hsa-miR-519c-3p | 3.55 | AKT1, CDKN1A, CREB1, ESR1, HDAC4, KDM6B, |
| | | PPARA, PRDM16, PTEN, RUNX2, SP1, STAT3 |
| hsa-miR520c-3p | 4.58 | AKT1, CDKN1A, ESR1, HDAC4, KDM6B, KLF6, |
| | | MECP2, PPARA, PPARGC1B, PRDM16, PTEN, |
| | | RUNX2, SP1, STAT3 |
| hsa-miR-93-5p | 3.01 | AKT1, CDKN1A, ESR1, KDM6B, KLF6, MECP2, |
| | | PPARA, PPARGC1B, PRDM16, PTEN, SIRT1, STAT3 |
| hsa-miR-520a-3p | 3.95 | AKT1, CREB1, ESR1, HDAC4, KDM6B, KLF6, MECP2, |
| | | PPARGC1B, PRDM16, PTEN, RUNX2, SOD2, STAT3 |
| hsa-miR-1470 | 4.35 | AKT1, CREB1, HDAC4, KDM6B, KLF6, MAPK14, |
| | | MECP2, PPARA, PPARGC1B, PTEN, RUNX2, SOD2, |
| | | STAT3 |
| hsa-miR-5089-3p | 4.29 | AKT1, CREB1, KLF6, MAPK14, MECP2, PRDM16, |
| | | PTEN, SIRT1, SOD2, STAT3 |
| hsa-miR-520b | 3.83 | AKT1, ESR1, HDAC4, KDM6B, KLF6, MECP2, PPARA, |
| | | PPARGC1B, PRDM16, PTEN, RUNX2, SOD2, STAT3 |

The inventors noted that, as these genes were required for normal metabolic functions, and miR-22-3p was likely to induce degradation and/or reduce the translation of these genes, a strategy involving antagonizing the functions of miR-22-3p could constitute a viable therapeutic route to the amelioration of metabolic disorders.

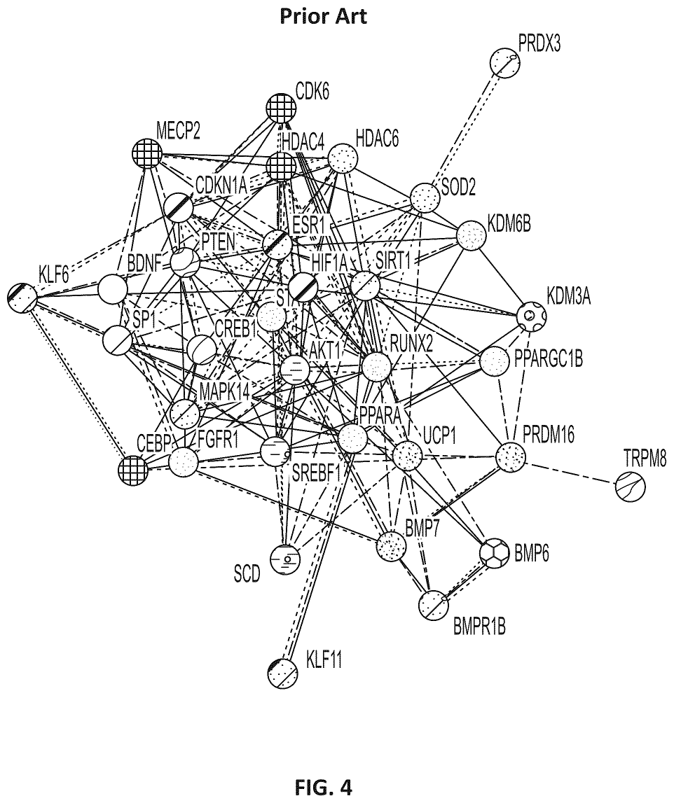

The Inventor further used the protein-protein interaction functional enrichment analysis tool STRING (https://string-db.org) to illustrate the various interactions within a network of 34 proteins related to miR-22 (see e.g., FIG. 4).

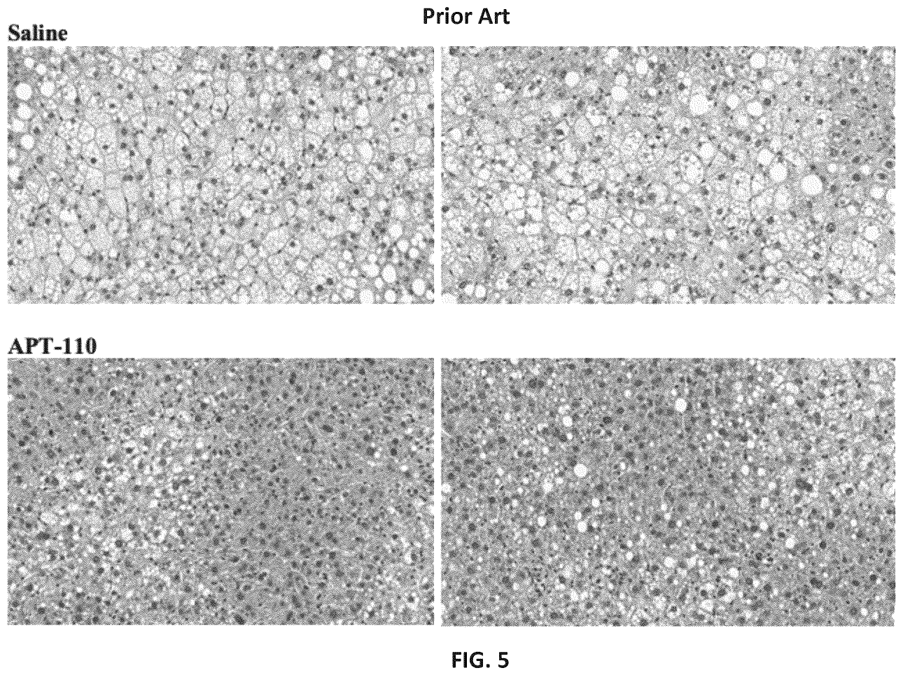

The inventors then proceeded to explore the metabolic effects of miR-22-3p antagomirs in vitro in primary cultures of human adipocytes and in vivo in mice. The metabolic and energetic benefits of the inventors first-generation of miR-22-3p antagomirs were summarized in two peer-reviewed articles published in 2020 [26, 38], both of which are incorporated herein in their entirety for the purposes described herein. In vivo proof of concept of miR-22-3p inhibition in mice was performed in the mouse model of diet-induced obesity (DIO) in C57BL/6J male mice. Mice of various ages were allocated to normal chow (10% fat) or a 60% high-fat diet, and mice were treated for up to 12 weeks with a miR-22-3p antagomir or saline. In the miR-22-3p antagomir treatment animals, the inventors consistently observed a reduction in body weight and fat mass (without alteration of lean mass); an improvement in glucose, insulin sensitivity and lipid profile; an increase in thermogenesis; and no modification of food intake or body temperature. Furthermore, treatment for 12 weeks with AptamiR's miR-22-3p antagomir, APT-110, produced a marked reduction in fatty infiltration of the liver (see e.g., FIG. 5).

### IV. SELECTION OF FAT FOR ACTIVE TARGETING DELIVERY OF MIRNA ONTS TO ADIPOCYTES AND/OR METABOLIC ORGANS

The Inventor selected the membrane transporter Fatty Acid Translocase (FAT) to facilitate active targeting delivery of miRNA ONTs to tissues of interest. FAT is the main route of uptake by adipose tissues of long-chain fatty acids. FAT also facilitates uptake by adipose tissues of short peptides, such as but not limited to, hexarelin, prohibitin, and/or thrombospondin peptide-1.

FAT is significantly expressed in cells and tissues sensitive to metabolic dysfunctions, such as adipocytes, hepatocytes, skeletal and cardiac myocytes, pancreatic β-cells, kidney glomeruli and tubules cells, monocytes, and macrophages. FAT cycles between the adipocyte membrane and intra-cellular compartments (endosomes).

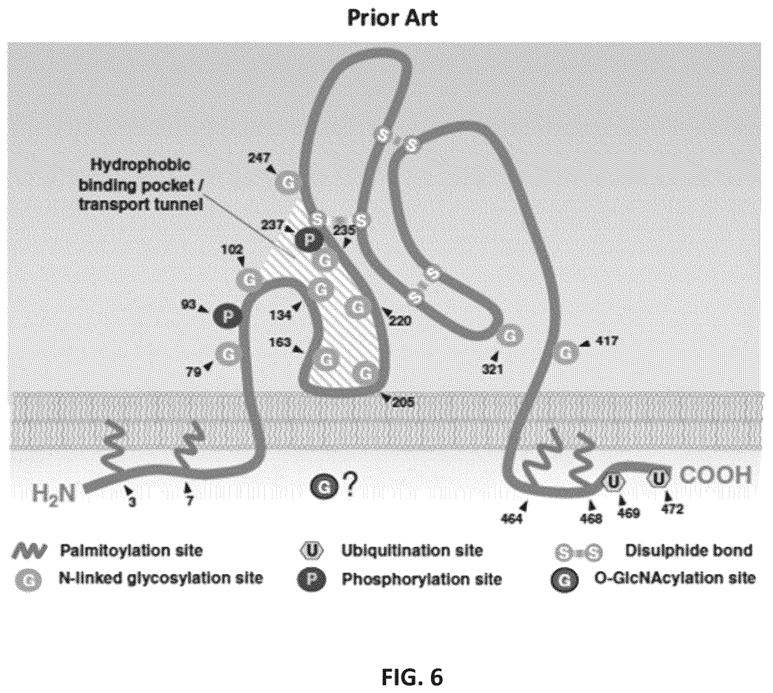

Cell surface proteins can cycle intra-cellularly, and many surface receptors and transporters are actively internalized in response to ligand binding. For example, FAT is an integral membrane glycoprotein made of a single chain of 472 amino acids (53 kDa) that has a hairpin membrane topology with two transmembrane spanning regions, with both the NH₂ and COOH termini as short segments in the cellular cytoplasm [39-41], (see e.g., FIG. 6, adapted from [41]).

### V. DESIGN OF GENERATION 2.5 MIR-22-3P ANTAGOMIRS

Pre-IND toxicology and safety studies of the Inventors first-generation miR-22-3p antagomir APT-110 (a “naked” single-stranded 18-mer miR-22-3p antagomir containing PS and LNA modifications) were completed in mice and non-human primates according to FDA guidance. In non-human primates, a transient activation of blood platelets and of the complement pathway was observed right after subcutaneous administration of supra-therapeutic doses. Kidney and liver histologic alterations were also noted. Due to these observations, the Inventors first-generation compounds containing phosphorothioate (PS) and Locked Nucleic Acid (LNA) chemical modifications were not developed further. Therefore, the inventors designed and implemented new and innovative Generation 2.5 miR-22-3p Antagomirs that comprised a miR-22-3p antagomir linked to a lipid or peptide targeting agent.

To design Generation 2.5 miR-antagomirs, the inventors implemented the following selection criteria/modifications: a) eliminate potential toxicities by replacing PS and LNA chemical modifications with a PNA backbone (e.g., a gamma PNA backbone); b) maintain resistance to nucleases and proteases/peptidases; c) avoid chirality; d) limit binding to serum proteins; e) improve and/or simplify chemical synthesis; and e) conjugate ONT to a fatty acid and/or a short peptide for enhanced targeted delivery of therapeutic agents to adipocytes and/or metabolic organs (e.g., to facilitate significant reduction in required effective doses and/or increase duration of action (e.g., mean residence time)).

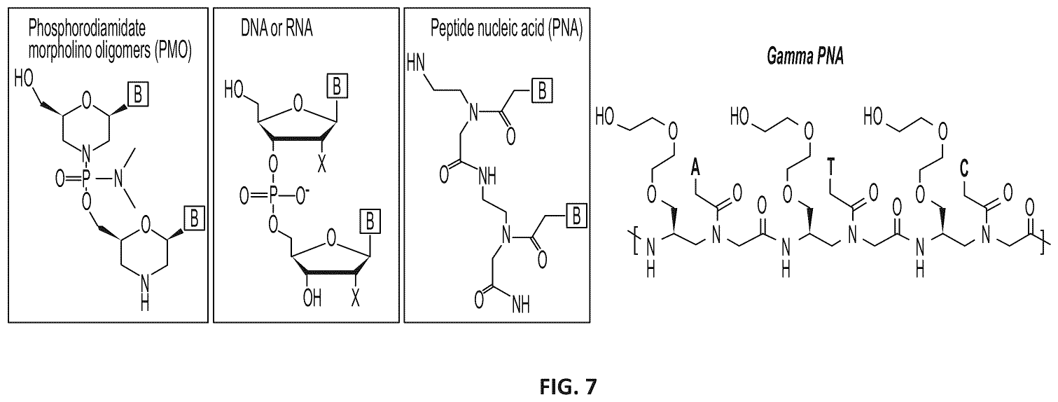

The Inventors designed miRNA ONTs with a peptide nucleic acid (PNA) backbone (see e.g., FIG. 7). In PNA compounds, the sugar-backbone of an oligonucleotide is replaced with an amide containing backbone, for example but not limited to, an aminoethylglycine backbone. The nucleobases can be retained and are bound directly or indirectly to aza nitrogen atoms of the amide portion of the backbone. In some embodiments, neutral pseudopeptide backbone of PNAs contributes to enhanced binding affinity and/or high biological stability. In some embodiments, PNAs hybridize with the complementary site in a target RNA and act by a steric hindrance-based mechanism. In some embodiments, γPNA modifications can transform a randomly folded PNA into a helical structure. In some embodiments, incorporation of an L-serine side chain at the γ-position induces the preorganization of PNA into a right-handed helix and improves the hybridization stability with the target. Representative United States patents that teach the preparation of PNA compounds comprise, but are not limited to, U.S. Pat. Nos. 5,539,082; 5,714,331; and 5,719,262, each of which is herein incorporated by reference for the purposes described herein. Further teaching of PNA compounds can be found in Nielsen et al., Science, 1991, 254, 1497-1500, which is incorporated herein in its entirety for the purposes described herein.

In some embodiments, a PNA backbone is very stable and/or is not degraded by naturally occurring nucleases and/or proteases. In some embodiments, a PNA can recognize its complementary nucleic acid strand with high affinity through nucleobase complementary. In some embodiments, a PNA recognizes its complementary nucleic acid strand with excellent specificity that surpasses the specificity of natural nucleic acids and/or certain of their analogs [42].

In some embodiments, a drawback with using PNA is that it poorly penetrates cells when utilized alone. However, in some embodiments, PNA can enter cells effectively when coupled with other molecules, such as but not limited to lipids and/or peptides that can readily be taken up by cells [43].

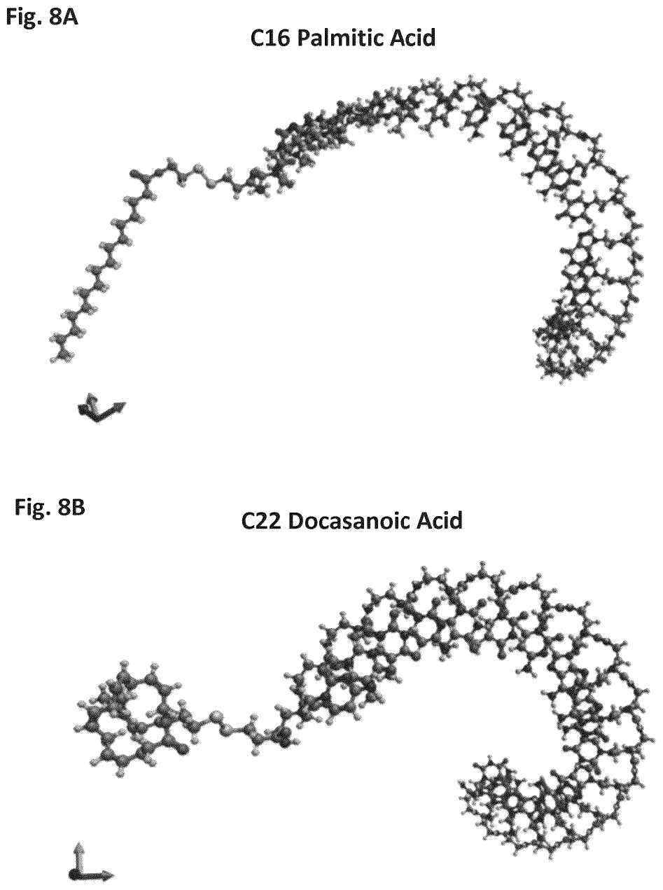

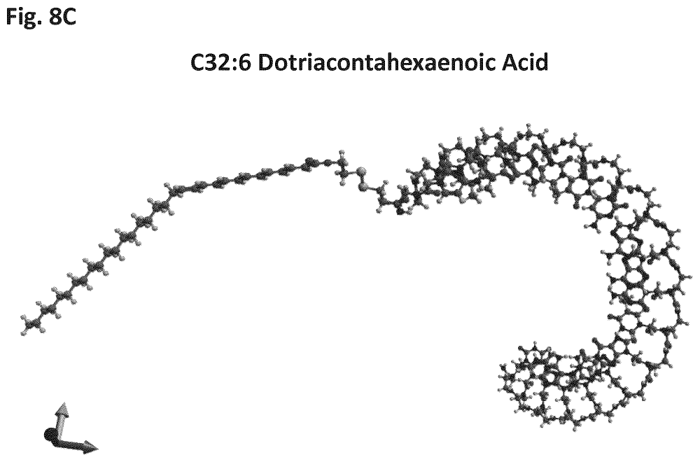

Using high-performance molecular dynamics modeling on graphics processing units, the inventors created molecular models of Generation 2.5 miR-22-3p antagomirs, coupled with a fatty acid. As shown in FIG. 8, the fatty acids can be of increasing length, for example, C16 palmitic acid, to C22:6 docosahexaenoic acid, and to C32:6 dotriacontahexaenoic acid (see e.g., FIG. 8)

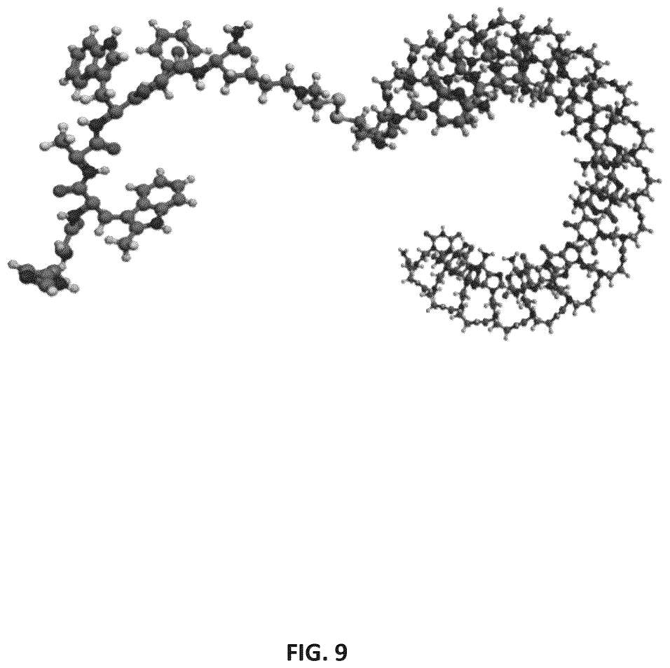

Using high-performance molecular dynamics modeling on graphics processing units, the inventors also created molecular models of Generation 2.5 miR-22-3p antagomirs, coupled to a peptide like Hexarelin (His-D-2-Me-Trp-Ala-Trp-D-Phe-Lys-NH2) (see e.g., FIG. 9).

### VI. REFERENCES

The following references, and those cited elsewhere herein, to the extent that they provide exemplary procedural or other details supplementary to those set forth herein, are specifically incorporated herein by reference.
1. Correa, L. H., G. S. Heyn, and K. G. Magalhaes, The Impact of the Adipose Organ Plasticity on Inflammation and Cancer Progression. Cells, 2019. 8(7).
2. Wilding, J. P. H., et al., Once-Weekly Semaglutide in Adults with Overweight or Obesity. N Engl J Med, 2021. 384(11): p. 989-1002.
3. Jastreboff, A. M., et al., Tirzepatide Once Weekly for the Treatment of Obesity. N Engl J Med, 2022. 387(3): p. 205-216.
4. le Roux, C. W., et al., Tirzepatide for the treatment of obesity: Rationale and design of the SURMOUNT clinical development program. Obesity (Silver Spring), 2023. 31(1): p. 96-110.
5. Younossi, Z., et al., Global burden of NAFLD and NASH: trends, predictions, risk factors and prevention. Nat Rev Gastroenterol Hepatol, 2018. 15(1): p. 11-20.
6. Hanson, A., D. Wilhelmsen, and J. K. DiStefano, The Role of Long Non-Coding RNAs (lncRNAs) in the Development and Progression of Fibrosis Associated with Nonalcoholic Fatty Liver Disease (NAFLD). Noncoding RNA, 2018. 4(3).
7. Sarwar, R., N. Pierce, and S. Koppe, Obesity and nonalcoholic fatty liver disease: current perspectives. Diabetes Metab Syndr Obes, 2018. 11: p. 533-542.
8. Buzzetti, E., M. Pinzani, and E. A. Tsochatzis, The multiple-hit pathogenesis of non-alcoholic fatty liver disease (NAFLD). Metabolism, 2016. 65(8): p. 1038-48.
9. Iacomino, G. and A. Siani, Role of microRNAs in obesity and obesity-related diseases. Genes Nutr, 2017. 12: p. 23.
10. Moumne, L., A. C. Marie, and N. Crouvezier, Oligonucleotide Therapeutics: From Discovery and Development to Patentability. Pharmaceutics, 2022. 14(2).
11. Iacomino, G., miRNAs: The Road from Bench to Bedside. Genes (Basel), 2023. 14(2).
12. van Rooij, E. and S. Kauppinen, Development of microRNA therapeutics is coming of age. EMBO Mol Med, 2014. 6(7): p. 851-64.
13. Li, Z. and T. M. Rana, Therapeutic targeting of microRNAs: current status and future challenges. Nat Rev Drug Discov, 2014. 13(8): p. 622-38.
14. Zhang, Y., Z. Wang, and R. A. Gemeinhart, Progress in microRNA delivery. J Control Release, 2013. 172(3): p. 962-74.
15. Wittrup, A. and J. Lieberman, Knocking down disease: a progress report on siRNA therapeutics. Nat Rev Genet, 2015. 16(9): p. 543-52.
16. Nikam, R. R. and K. R. Gore, Journey of siRNA: Clinical Developments and Targeted Delivery. Nucleic Acid Ther, 2018. 28(4): p. 209-224.
17. Hammond, S. M., et al., Delivery of oligonucleotide-based therapeutics: challenges and opportunities. EMBO Mol Med, 2021. 13(4): p. e13243.
18. Roberts, T. C., R. Langer, and M. J. A. Wood, Advances in oligonucleotide drug delivery. Nat Rev Drug Discov, 2020. 19(10): p. 673-694.
19. Dowdy, S. F., et al., Delivery of RNA Therapeutics: The Great Endosomal Escape! Nucleic Acid Ther, 2022. 32(5): p. 361-368.
20. Zhang, C. and B. Zhang, RNA therapeutics: updates and future potential. Sci China Life Sci, 2023. 66(1): p. 12-30.
21. Alexander, R., H. Lodish, and L. Sun, MicroRNAs in adipogenesis and as therapeutic targets for obesity. Expert opinion on therapeutic targets, 2011. 15: p. 623-36.
22. Rupert, J. E. and M. G. Kolonin, Fatty acid translocase: a culprit of lipid metabolism dysfunction in disease. Immunometabolism (Cobham), 2022. 4(3): p. e00001.
23. Gutierrez, L. S. and J. Gutierrez, Thrombospondin 1 in Metabolic Diseases. Front Endocrinol (Lausanne), 2021. 12: p. 638536.
24. Marechal, L., et al., The CD36-PPARgamma Pathway in Metabolic Disorders. Int J Mol Sci, 2018. 19(5).
25. Thibonnier, M. and S. Ghosh, Strategy for Pre-Clinical Development of Active Targeting MicroRNA Oligonucleotide Therapeutics for Unmet Medical Needs. Int J Mol Sci, 2023. 24(8).
26. Thibonnier, M., et al., Metabolic and energetic benefits of microRNA-22 inhibition. BMJ Open Diabetes Res Care, 2020. 8(1).
27. Glatz, J. F., et al., Regulation of the subcellular trafficking of CD36, a major determinant of cardiac fatty acid utilization. Biochim Biophys Acta, 2016. 1861(10): p. 1461-71.
28. Inui, M., G. Martello, and S. Piccolo, MicroRNA control of signal transduction. Nat Rev Mol Cell Biol, 2010. 11: p. 252-63.
29. Ha, M. and V. N. Kim, Regulation of microRNA biogenesis. Nat Rev Mol Cell Biol, 2014. 15(8): p. 509-24.
30. Matsui, M., T. P. Prakash, and D. R. Corey, Argonaute 2-dependent Regulation of Gene Expression by Single-stranded miRNA Mimics. Mol Ther, 2016. 24(5): p. 946-55.
31. Chorn, G., et al., Single-stranded microRNA mimics. RNA, 2012. 18(10): p. 1796-804.
32. Lima, W. F., et al., Single-stranded siRNAs activate RNAi in animals. Cell, 2012. 150(5): p. 883-94.
33. Hu, J., et al., Exploring the effect of sequence length and composition on allele-selective inhibition of human huntingtin expression by single-stranded silencing RNAs. Nucleic Acid Ther, 2014. 24(3): p. 199-209.
34. Traube, F. R. and T. Carell, The chemistries and consequences of DNA and RNA methylation and demethylation. RNA Biol, 2017. 14(9): p. 1099-1107.
35. Tateishi, K., et al., Role of Jhdm2a in regulating metabolic gene expression and obesity resistance. Nature, 2009. 458(7239): p. 757-61.
36. Okada, Y., K. Tateishi, and Y. Zhang, Histone demethylase JHDM2A is involved in male infertility and obesity. Journal of andrology, 2010. 31(1): p. 75-8.
37. Abe, Y., et al., JMJD1A is a signal-sensing scaffold that regulates acute chromatin dynamics via SWI/SNF association for thermogenesis. Nat Commun, 2015. 6: p. 7052.
38. Thibonnier, M. and C. Esau, Metabolic Benefits of MicroRNA-22 Inhibition. Nucleic Acid Ther, 2020. 30(2): p. 104-116.
39. Glatz, J. F. and J. J. Luiken, From fat to FAT (CD36/SR-B2): Understanding the regulation of cellular fatty acid uptake. Biochimie, 2017. 136: p. 21-26.
40. Zhou, D., et al., CD36 level and trafficking are determinants of lipolysis in adipocytes. FASEB J, 2012. 26(11): p. 4733-42.
41. Luiken, J. J., et al., Post-translational modifications of CD36 (SR-B2): Implications for regulation of myocellular fatty acid uptake. Biochim Biophys Acta, 2016. 1862(12): p. 2253-2258.
42. Suparpprom, C. and T. Vilaivan, Perspectives on conformationally constrained peptide nucleic acid (PNA): insights into the structural design, properties and applications. RSC Chem Biol, 2022. 3(6): p. 648-697.
43. Yavari, N., L. Goltermann, and P. E. Nielsen, Uptake, Stability, and Activity of Antisense Anti-acpP PNA-Peptide Conjugates in Escherichia coli and the Role of SbmA. ACS Chem Biol, 2021. 16(3): p. 471-479.

1. A composition comprising,
a) one or more miRNA oligonucleotide therapeutic (ONT) agent(s); and
b) one or more targeting element(s) linked to the ONT by a linker, wherein the one or more targeting element(s) facilitate active targeted cellular uptake and/or delivery of the ONT agent to adipocytes and/or metabolic organs.

2. The composition of claim 1, wherein the one or more ONT agent(s) comprise miRNA agomirs, and/or antagomirs targeting specific miRNAs.

3. The composition of claim 1, wherein the one or more ONT agent(s) are 14 to 25 nucleotides in length.

4. The composition of claim 1, wherein one or more of the ONT agent(s) comprise a peptide nucleic acid (PNA) backbone.

5. The composition of claim 1, wherein the ONT agent comprises a miR-22 antagomir and/or a miR-515 agomir.

6. The composition of claim 1, wherein the ONT agent comprises a miR-22 antagomir according to SEQ ID NO: 12 (5′-CTTCTTCAACTGGCAGCT-3′), and/or a miR-515 agomir according to SEQ ID NO: 13 (5′-GAGUGCCUUCUUUUGGAGCGUU-3′).

7. The composition of claim 1, wherein the one or more targeting element(s) comprise a lipid.

8. The composition of claim 7, wherein the lipid comprises, cholesterol, decanoic acid, dodecanoic acid, palmitic acid, stearic acid, oleic acid/hexadecanoic acid, oleoyl glycine, docosanoic acid, dotriacontahexaenoic acid, and/or docosahexaenoic acid.

9. The composition of claim 7, wherein the lipid comprises a fatty acid that is transported by the membrane transporter Fatty Acid Translocase (FAT)).

10. The composition of claim 1, wherein the ONT agent is linked to one or more targeting elements by a linker selected from the group consisting of a covalent bond, a disulfide bond, a diester bond, a peptide bond, an ionic bond, and a biotin-streptavidin bond.

11. The composition of claim 1, wherein a linker is a cleavable linker.

12. The composition of claim 1, wherein one or more targeting element(s) specifically bind to the membrane transporter FAT.

13. The composition of claim 1, wherein the one or more targeting element(s) comprises a peptide.

14. The composition of claim 13, wherein the peptide comprises Hexarelin having the amino acid sequence His-D-2-methyl-Trp-Ala-Trp-D-Phe-Lys-NH2 (SEQ ID NO: 4), Thrombospondin-1 (TSP-1), a TSP-1 peptide having the amino acid sequence GVITRIR (SEQ ID NO: 1) and/or VTCGVITRIR (SEQ ID NO: 2), and/or a Prohibitin (PHB) peptide having the amino acid sequence CKGGRAKDC (SEQ ID NO: 3).

15. The composition of claim 13, wherein the one or more ONT agent(s) is linked to a peptide targeting element by a linker selected from the group consisting of a covalent bond, a disulfide bond, a diester bond, a peptide bond, an ionic bond, and a biotin-streptavidin bond.

16. The composition of claim 15, wherein a linker is a cleavable linker.

17. The composition of claim 1, wherein the one or more ONT agent(s) modulate lipid oxidation, mitochondrial activity, energy expenditure, fat accumulation, inflammation, and/or necrosis.

18. A method of treating a subject comprising administration of the composition according to claim 1.

19. The method of claim 18, wherein administering the composition comprises subcutaneous, transcutaneous, and/or intravenous administration.

20. The method of claim 18, wherein the subject has or is at risk of developing obesity and/or a cardio-metabolic disorder.

21. The method of claim 20, wherein the subject has or is at risk of developing dyslipidemia, Type 2 diabetes mellitus, Non-Alcoholic Fatty Liver Disease (NAFLD), Non Alcoholic Steatohepatitis (NASH), and/or Metabolic Associated Fatty Liver Disease (MAFLD).


SEQUENCE LISTING (13 sequences)

SEQ ID NO: 1 - protein, 7 aa, synthetic construct
GVITRIR

SEQ ID NO: 2 - protein, 10 aa, synthetic construct
VTCGVITRIR

SEQ ID NO: 3 - protein, 9 aa, synthetic construct
CKGGRAKDC

SEQ ID NO: 4 - protein, 6 aa, synthetic construct
  source 1..6: note = His-D-2-Me-Trp-Ala-Trp-D-Phe-Lys-NH2
  MOD_RES 2: note = 2-Methyl-D-Tryptophan
  MOD_RES 5: note = D-Phenylalanine
HWAWFK

SEQ ID NO: 5 - RNA, 23 nt, Homo sapiens
  source 1..23: mol_type = mRNA
tggaaatgaattacaggcagctg

SEQ ID NO: 6 - RNA, 23 nt, Homo sapiens
  source 1..23: mol_type = mRNA
cccttccaccccattggcagctc

SEQ ID NO: 7 - RNA, 23 nt, Homo sapiens
  source 1..23: mol_type = mRNA
aaatggcagacctccggcagctg

SEQ ID NO: 8 - RNA, 23 nt, Homo sapiens
  source 1..23: mol_type = mRNA
atgagtggatggagtgcagctaa

SEQ ID NO: 9 - RNA, 23 nt, Homo sapiens
  source 1..23: mol_type = mRNA
gctgcatatgtttagggcagctt

SEQ ID NO: 10 - RNA, 23 nt, Homo sapiens
  source 1..23: mol_type = mRNA
attttccaaaactgtggcagcta

SEQ ID NO: 11 - RNA, 22 nt, Homo sapiens
  source 1..22: mol_type = other RNA
tgtcaagaagttgaccgtcgaa

SEQ ID NO: 12 - RNA, 18 nt, synthetic construct
  source 1..18: mol_type = other RNA
cttcttcaactggcagct

SEQ ID NO: 13 - RNA, 22 nt, synthetic construct
  source 1..22: mol_type = other RNA
gagtgccttcttttggagcgtt